(19) United States
(12) Patent Application Publication
(10) Pub. No.: US 2003/0212076 A1
(43) Pub. Date: Nov. 13, 2003

(54) Fibrinogen receptor antagonists

(75) Inventors: Dirk A. Heerding, Malvern, PA (US); James Martin Samanen, Phoenixville, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/410,826

(22) Filed: Apr. 10, 2003

Related U.S. Application Data
(63) Continuation of application No. 09/996,136, filed on Nov. 28, 2001, now abandoned.
Application No. 09/996,136 is a continuation of application No. 09/840,358, filed on Apr. 23, 2001, now abandoned.
Application No. 09/840,358 is a continuation of application No. 09/445,489, filed on Dec. 8, 1999, now abandoned.
Application No. 09/445,489 is a 371 of international application No. PCT/US98/15288, filed on Jul. 23, 1998.
(60) Provisional application No. 60/063,545, filed on Oct. 28, 1997.
(60) Provisional application No. 60/053,788, filed on Jul. 25, 1997.

(51) Int. Cl. A61K 31/496; C 07D 4 3/02

(52) U.S. Cl. 514/253.12; 544/360

(57) ABSTRACT

This invention relates to a compound of the formula (I):

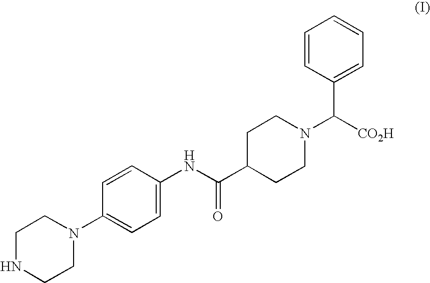

or a pharmaceutically acceptable salt thereof, which is effective for inhibiting platelet aggregation, pharmaceutical compositions for effecting such activity, and a method for inhibiting platelet aggregation.

### FIELD OF THE INVENTION

This invention relates to novel compounds which inhibit platelet aggregation, pharmaceutical compositions containing these compounds and methods of using the compounds.

### BACKGROUND OF THE INVENTION

Platelet aggregation is believed to be mediated primarily through the fibrinogen receptor, or GPIIb-IIIa platelet receptor complex, which is a member of a family of adhesion receptors referred to as integrins. It has been found that frequently the natural ligands of integrin receptors are proteins which contain an Arg-Gly-Asp sequence. Von Willebrand factor and fibrinogen, which are considered to be natural ligands for the GPIIb-IIIa receptor, possess an Arg-Gly-Asp (RGD in single letter amino acid code) sequence in their primary structure. Functionally, these proteins are able to bind and crosslink GPIIb-IIIa receptors on adjacent platelets and thereby effect aggregation of platelets.

Fibronectin, vitronectin and thrombospondin are RGD-containing proteins which have also been demonstrated to bind to GPIIb-IIIa. Fibronectin is found in plasma and as a structural protein in the intracellular matrix. Binding between the structural proteins and GPIIb-IIIa may function to cause platelets to adhere to damaged vessel walls.

Linear and cyclic peptides which bind to vitronectin and contain an RGD sequence are disclosed in WO 89/05150 (PCT US88/04403). EP 0 275 748 discloses linear tetra- to hexapeptides and cyclic hexa- to octapeptides which bind to the GPIIb-IIIa receptor and inhibit platelet aggregation. Other linear and cyclic peptides, the disclosure of which are incorporated herein by reference, are reported in EP-A 0 341 915. However, the peptide like structures of such inhibitors often pose problems, such as in drug delivery, metabolic stability and selectivity. Inhibitors of the fibrinogen receptor which are not constructed of natural amino acid sequences are disclosed in EP-A 0 372,486, EP-A 0 381 033 and EP-A 0 478 363. WO 92/07568 (PCT/US91/08166) discloses fibrinogen receptor antagonists which mimic a conformational γ-turn in the RGD sequence by forming a monocyclic seven-membered ring structure. There remains a need, however, for novel fibrinogen receptor antagonists (e.g., inhibitors of the GPIIb-IIIa protein) which have potent in vivo and in vitro effects and lack the peptide backbone structure of amino acid sequences.

The present invention discloses novel compounds. These compounds inhibit the GPIIb-IIIa receptor and inhibit platelet aggregation.

### SUMMARY OF THE INVENTION

In one aspect this invention is a compound as described hereinafter in formula (I).

This invention is also a pharmaceutical composition for inhibiting platelet aggregation or clot formation, which comprises a compound of formula (I) and a pharmaceutically acceptable carrier.

This invention is further a method for inhibiting platelet aggregation in a mammal in need thereof, which comprises internally administering an effective amount of a compound of formula (I).

In another aspect, this invention provides a method for inhibiting reocclusion of an artery or vein in a mammal following fibrinolytic therapy, which comprises internally administering an effective amount of a fibrinolytic agent and a compound of formula (I). This invention is also a method for treating stroke, transient ischemia attacks, or myocardial infarction.

### DETAILED DESCRIPTION OF THE INVENTION

This invention discloses a compound which inhibits platelet aggregation. The compound of the instant invention is believed to interact favorably with the GPIIb-IIIa receptor.

Although not intending to be bound to any specific mechanism of action, this compound is believed to inhibit the binding of fibrinogen to the platelet-bound fibrinogen receptor GPIIb-IIIa, and may interact with other adhesion proteins via antagonism of a putative RGD binding site.

The compound of this invention is a compound of formula (I):

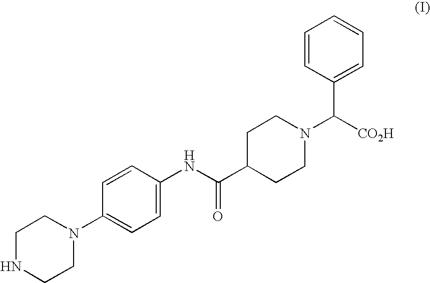

or a pharmaceutically acceptable salt thereof. This compound is 4-[(4-(1-piperizinyl)phenyl)aminocarbonyl]-1-piperidine-1-phenylacetic acid or a pharmaceutically acceptable salt thereof.

Also included in this invention are pharmaceutically acceptable addition salts, complexes or prodrugs of the compound of this invention. Prodrugs are considered to be any covalently bonded carriers which release the active parent drug according to formula (I) in vivo. Such prodrugs are, for example, compounds of formula (II):

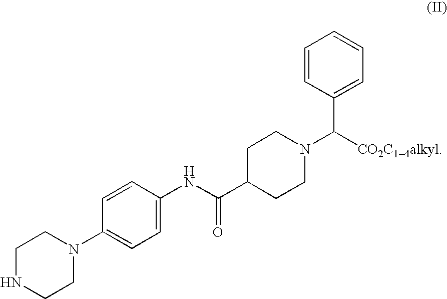

In cases wherein the compound of this invention may have one or more chiral centers, unless specified, this invention includes each unique nonracemic compound which may be synthesized and resolved by conventional techniques. The meaning of any substituent at any one occurrence is independent of its meaning, or any other substituent's meaning, at any other occurrence, unless specified otherwise.

The preferred compound of this invention is 4-[(4-( 1-piperizinyl)-phenyl)aminocarbonyl]-1-piperidine-1-phenylacetic acid or a pharmaceutically acceptable salt thereof.

This invention also includes the following compounds which are useful in the methods of the instant invention:

(−)-4-[(4-(1-piperizinyl)phenyl)aminocarbonyl]-1-piperidine-1-phenylacetic acid,

(+)-4-[(4-(1-piperizinyl)phenyl)aminocarbonyl]-1-piperidine-1-phenylacetic acid,

2-(4-[4-(1-piperazinyl)phenylamino]carbonylpiperadino)-2-(4-methoxyphenyl)acetic acid,

2-(4-[4-(1-piperazinyl)phenylamino]carbonylpiperadino)-2-(4-tert-butylphenyl)acetic acid,

2-(4-[4-(1-piperazinyl)phenylamino]carbonylpiperadino)butanoic acid,

3-methyl-2-(4-[4-(1-piperazinyl)phenylamino]carbonylpiperadino)butanoic acid, and

2-(4-[4-(1-piperazinyl)phenylamino]carbonylpiperadino)-2-(4-nitrophenyl)acetic acid;

or a pharmaceutically acceptable salt thereof.

Certain radical groups are abbreviated herein. t-Bu refers to the tertiary butyl radical, Boc refers to the t-butyloxycarbonyl radical, Ph refers to the phenyl radical, Bzl refers to the benzyl radical, Me refers to methyl, Et refers to ethyl, Ac refers to acetyl, Alk refers to C₁₋₆alkyl, Nph refers to 1- or 2-naphthyl and cHex refers to cyclohexyl.

Certain reagents are abbreviated herein. DMAP refers to dimethylaminopyridine, DIEA refers to diisopropylethyl amine, EDC refers to N-ethyl-N′(dimethylaminopropyl)-carbodiimide. HOBt refers to 1-hydroxybenzotriazole, THF refers to tetrahydrofuran, DMF refers to dimethyl formamide, Pd/C refers to a palladium on carbon catalyst, TEA refers to triethylamine, TFA refers to trifluoroacetic acid.

The compounds of formula (I) are generally prepared by reacting a compound of the formula (III) with a compound of the formula (IV):

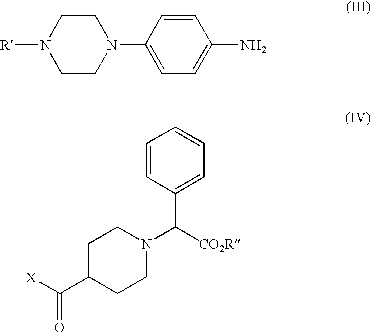

wherein R′ is an amine protecting group, R″ is a C₁₋₄alkyl group and X is OH or chloro;

and thereafter removing any protecting groups, and optionally forming a pharmaceutically acceptable salt.

The compound of formula (I) is prepared by the method described in Scheme I.

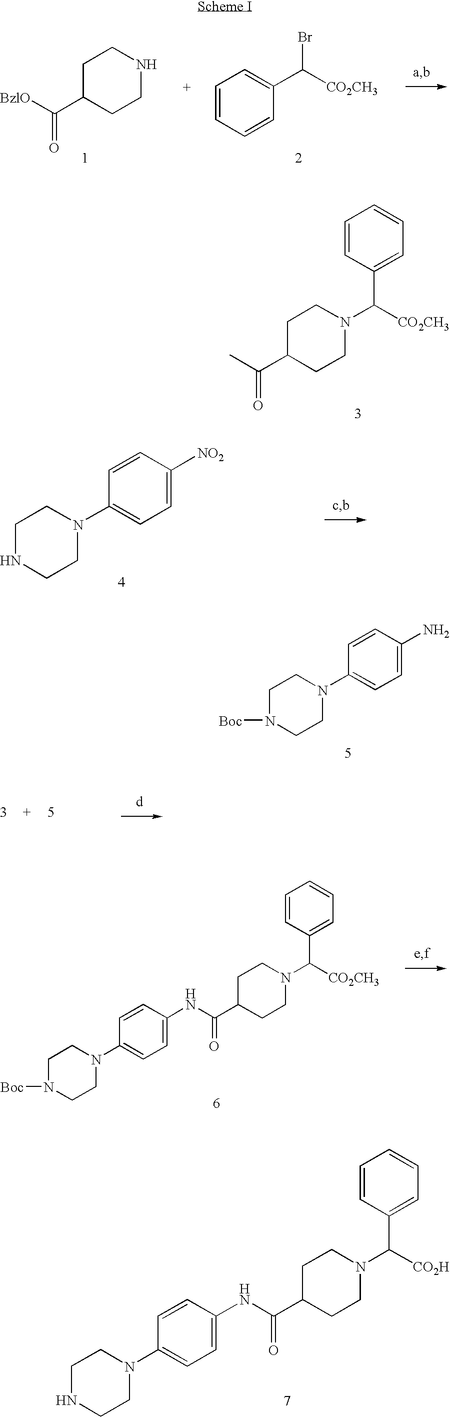

a) Et₃N, THF, reflux; b) H₂, 10% Pd/C, MeOH; c) (Boc)₂O; d) EDC, pyridine; e) NaOH, EtOH; f) TFA, CH₂Cl₂.

A suitably protected amine, such as I-1 is reacted with a commercially available suitable bromoester, such as I-2, in a polar aprotic solvent, such as THF, to give the substitution product. If neccessary, additional base, such as triethyl amine, can be added to reaction mixture to nuetralize the acid produced in the substitution reaction. Additionally, the reaction may heated to reflux to increase the rate of reaction. The benzyl ester is removed by standard methods, such as hydrogenation over palladium, to give the corresponding carboxylic acid I-3.

A commercially available suitable amine, such as I-4, is protected on nitrogen with a suitable protecting group, such as t-butoxycarbonyl, in a suitable solvent, such as THF. Many other protecting group schemes can be devised for this compound and can be found in such volumes as Greene, “Protective Groups in Organic Synthesis” (published by Wiley-Interscience). The nitro group is then reduced by standard methods, such as hydrogenation over palladium catalyst, to give the corresponding amine I-5.

The carboxylic acid I-3 is activated in situ by standard methods, such as EDC, and reacted with amine I-5 in a suitable solvent, such as pyridine, to give the resulting amide I-6. Many other methods of affecting this transformation are known and can be found in such reference volumes, such as Larock, “Comprehensive Organic Transformations” (published by VCH Publishers). Saponification of the methyl ester is carried out using a base, such as NaOH, in a suitable polar solvent, such as EtOH,to give the corresponding carboxylic acid. Removal of the t-butoxycarbonyl group from nitrogen is accomplished by strong acid, such as trifluoroacetic acid, in a suitable solvent, such as CH₂Cl₂, to give the final compound I-7.

The compounds of the instant invention are prepared as described in Scheme I or by processes analogous to those described described in Scheme I. Furthermore, the compounds of the instant invention are prepared as described in Examples 1-8 hereinafter.

Acid addition salts of the compound of this invention are prepared in a standard manner in a suitable solvent from the parent compound and an excess of an acid, such as hydrochloric, hydrobromic, sulfuric, phosphoric, acetic, maleic, succinic or methanesulfonic. The acetate salt form is especially useful. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li+, Na+, K+, Ca++, Mg++ and NH₄+ are specific examples of cations present in pharmaceutically acceptable salts.

This invention provides a pharmaceutical composition which comprises a compound according to formula (I) and a pharmaceutically acceptable carrier. Accordingly, the compound of formula (I) may be used in the manufacture of a medicament. Pharmaceutical compositions of the compound of formula (I) prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, the compound of this invention may be encapsulated, tableted or prepared in a emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compound of this invention may also be combined with excipients such as cocoa butter, glycerin, gelatin or polyethylene glycols and molded into a suppository.

The compound of this invention may be used in vitro to inhibit the aggregation of platelets in blood and blood products, e.g., for storage, or for ex vivo manipulations such as in diagnostic or research use.

This invention also provides a method of inhibiting platelet aggregation and clot formation in a mammal, especially a human, which comprises the internal administration of a compound of formula (I) and a pharmaceutically acceptable carrier. Indications for such therapy include acute myocardial infarction (AMI), deep vein thrombosis, pulmonary embolism, dissecting anurysm, transient ischemia attack (TIA), stroke and other infarct-related disorders, and unstable angina. Chronic or acute states of hyper-aggregability, such as disseminated intravascular coagulation (DIC), septicemia, surgical or infectious shock, post-operative and post-partum trauma, cardiopulmonary bypass surgery, incompatible blood transfusion, abruptio placenta, thrombotic thrombocytopenic purpura (TTP), snake venom and immune diseases, are likely to be responsive to such treatment. In addition, the compound of this invention may be useful in a method for the prevention of metastatic conditions, the prevention or treatment of fungal or bacterial infection, inducing immunostimulation, treatment of sickle cell disease, and the prevention or treatment of diseases in which bone resorption is a factor.

The compound of formula (I) is administered either orally or parenterally to the patient, in a manner such that the concentration of drug in the plasma is sufficient to inhibit platelet aggregation, or other such indication. The pharmaceutical composition containing the compound is administered at a dose between about 0.2 to about 50 mg/kg in a manner consistent with the condition of the patient. For acute therapy, parenteral administration is preferred. For persistent states of hyperaggregability, an intravenous infusion of the peptide in 5% dextrose in water or normal saline is most effective, although an intramuscular bolus injection may be sufficient.

For chronic, but noncritical, states of platelet aggregability, oral administration of a capsule or tablet, or a bolus intramuscular injection is suitable. The compound of this invention is administered one to four times daily at a level of about 0.4 to about 50 mg/kg to achieve a total daily dose of about 0.4 to about 200 mg/kg/day.

This invention further provides a method for inhibiting the reocclusion of an artery or vein following fibrinolytic therapy, which comprises internal administration of a compound of formula (I) and a fibrinolytic agent. It has been found that administration of an peptide in fibrinolytic therapy either prevents reocclusion completely or prolongs the time to reocclusion.

When used in the context of this invention the term fibrinolytic agent is intended to mean any compound, whether a natural or synthetic product, which directly or indirectly causes the lysis of a fibrin clot. Plasminogen activators are a well known group of fibrinolytic agents. Useful plasminogen activators include, for example, anistreplase, urokinase (UK), pro-urokinase (pUK), streptokinase (SK), tissue plasminogen activator (tPA) and mutants, or variants, thereof, which retain plasminogen activator activity, such as variants which have been chemically modified or in which one or more amino acids have been added, deleted or substituted or in which one or more or functional domains have been added, deleted or altered such as by combining the active site of one plasminogen activator with the fibrin binding domain of another plasminogen activator or fibrin binding molecule. Other illustrative variants include tPA molecules in which one or more glycosylation sites have been altered. Preferred among plasminogen activators are variants of tPA in which the primary amino acid sequence has been altered in the growth factor domain so as to increase the serum half-life of the plasminogen activator. tPA Growth factor variants are disclosed, e.g., by Robinson et al., EP-A 0 297 589 and Browne et al., EP-A 0 240 334. Other variants include hybrid proteins, such as those disclosed in EP 0 028 489, EP 0 155 387 and EP 0 297 882, all of which are incorporated herein by reference. Anistreplase is a preferred hybrid protein for use in this invention. Fibrinolytic agents may be isolated from natural sources, but are commonly produced by traditional methods of genetic engineering.

Useful formulations of tPA, SK, UK and pUK are disclosed, for example, in EP-A 0 211 592, EP-A 0 092 182 and U.S. Pat. No. 4,568,543, all of which are incorporated herein by reference. Typically the fibrinolytic agent may be formulated in an aqueous, buffered, isotonic solution, such as sodium or ammonium acetate or adipate buffered at pH 3.5 to 5.5. Additional excipients such as polyvinyl pyrrolidone, gelatin, hydroxy cellulose, acacia, polyethylene, glycol, mannitol and sodium chloride may also be added. Such a composition can be lyophilized.

The pharmaceutical composition may be formulated with both the compound of formula (I) and fibrinolytic in the same container, but formulation in different containers is preferred. When both agents are provided in solution form they can be contained in an infusion/injection system for simultaneous administration or in a tandem arrangement.

Indications for such therapy include myocardial infarction, deep vein thrombosis, pulmonary embolism, stroke and other infarct-related disorders. The compound of formula (I) is administered just prior to, at the same time as, or just after parenteral administration of tPA or other fibrinolytic agent. It may prove desirable to continue treatment with the peptide for a period of time well after reperfusion has been established to maximally inhibit post-therapy reocclusion. The effective dose of tPA, SK, UK or pUK may be from 0.5 to 5 mg/kg and the effective dose of the compound of this invention may be from about 0.1 to 25 mg/kg.

For convenient administration of the inhibitor and the fibrinolytic agent at the same or different times, a kit is prepared, comprising, in a single container, such as a box, carton or other container, individual bottles, bags, vials or other containers each having an effective amount of the inhibitor for parenteral administration, as described above, and an effective amount of tPA, or other fibrinolytic agent, for parenteral administration, as described above. Such kit can comprise, for example, both pharmaceutical agents in separate containers or the same container, optionally as lyophilized plugs, and containers of solutions for reconstitution. A variation of this is to include the solution for reconstitution and the lyophilized plug in two chambers of a single container, which can be caused to admix prior to use. With such an arrangement, the fibrinolytic and the compound of this invention may be packaged separately, as in two containers, or lyophilized together as a powder and provided in a single container.

When both agents are provided in solution form, they can be contained in an infusion/injection system for simultaneous administration or in a tandem arrangement. For example, the platelet aggregation inhibitor may be in an i.v. injectable form, or infusion bag linked in series, via tubing, to the fibrinolytic agent in a second infusion bag. Using such a system, a patient can receive an initial bolus-type injection or infusion, of the inhibitor followed by an infusion of the fibrinolytic agent.

The pharmacological activity of the compound of this invention is assessed by its ability to inhibit the binding of ³H-SK&F 107260, a known RGD-fibrinogen antagonist, to the GPIIbIIIa receptor; its ability to inhibit platelet aggregation, in vitro, and its ability to inhibit thrombus formation in vivo.

### Inhibition of RGD-Mediated GPIIb-IIIa Binding

Purification of GPIIb-IIIa

Ten units of outdated, washed human platelets (obtained from Red Cross) were lyzed by gentle stirring in 3% octylglucoside, 20 mM Tris-HCl, pH 7.4, 140 mM NaCl, 2 mM CaCl₂ at 4° C. for 2 h. The lysate was centrifuged at 100,000 g for 1 h. The supernatant obtained was applied to a 5 mL lentil lectin sepharose 4B column (E.Y. Labs) preequilibrated with 20 mM Tris-HCl, pH 7.4, 100 mM NaCl, 2 mM CaCl₂, 1% octylglucoside (buffer A). After 2 h incubation, the column was washed with 50 mL cold buffer A. The lectin-retained GPIIb-IIIa was eluted with buffer A containing 10% dextrose. All procedures were performed at 4° C. The GPIIb-IIIa obtained was >95% pure as shown by SDS polyacrylamide gel electrophoresis.

Incorporation of GPIIb-IIIa in Liposomes.

A mixture of phosphatidylserine (70%) and phosphatidylcholine (30%) (Avanti Polar Lipids) were dried to the walls of a glass tube under a stream of nitrogen. Purified GPIIb-IIIa was diluted to a final concentration of 0.5 mg/mL and mixed with the phospholipids in a protein:phospholipid ratio of 1:3 (w:w). The mixture was resuspended and sonicated in a bath sonicator for 5 min. The mixture was then dialyzed overnight using 12,000-14,000 molecular weight cutoff dialysis tubing against a 1000-fold excess of 50 mM Tris-HCl, pH 7.4, 100 mM NaCl, 2 mM CaCl2 (with 2 changes). The GPIIb-IIIa-containing liposomes wee centrifuged at 12,000g for 15 min and resuspended in the dialysis buffer at a final protein concentration of approximately 1 mg/mL. The liposomes were stored at −70 C. until needed.

Competitive Binding to GPIIb-IIIa

The binding to the fibrinogen receptor (GPIIb-IIIa) was assayed by an indirect competitive binding method using [³H]-SK&F-107260 as an RGD-type ligand. The binding assay was performed in a 96-well filtration plate assembly (Millipore Corporation, Bedford, Mass.) using 0.22 um hydrophilic durapore membranes. The wells were precoated with 0.2 mL of 10 μg/mL polylysine (Sigma Chemical Co., St. Louis, Mo.) at room temperature for 1 h to block nonspecific binding. Various concentrations of unlabeled benzadiazapines were added to the wells in quadruplicate. [³H]-SK&F-107260 was applied to each well at a final concentration of 4.5 nM, followed by the addition of 1 μg of the purified platelet GPIIb-IIIa-containing liposomes. The mixtures were incubated for 1 h at room temperature. The GPIIb-IIIa-bound [3H]-SK&F-107260 was seperated from the unbound by filtration using a Millipore filtration manifold, followed by washing with ice-cold buffer (2 times, each 0.2 mL). Bound radioactivity remaining on the filters was counted in 1.5 mL Ready Solve (Beckman Instruments, Fullerton, Calif.) in a Beckman Liquid Scintillation Counter (Model LS6800), with 40% efficiency. Nonspecific binding was determined in the presence of 2 μM unlabeled SK&F-107260 and was consistently less than 0.14% of the total radioactivity added to the samples. All data points are the mean of quadruplicate determinations.

Competition binding data were analyzed by a nonlinear least-squares curve fitting procedure. This method provides the IC50 of the antagonists (concentration of the antagonist which inhibits specific binding of [³H]-SK&F-107260 by 50% at equilibrium). The IC50 is related to the equilibrium dissociation constant (Ki) of the antagonist based on the Cheng and Prusoff equation: Ki=IC50/(1+L/Kd), where L is the concentration of [3H]-SK&F-107260 used in the competitive binding assay (4.5 nM), and Kd is the dissociation constant of [3H]-SK&F-107260 which is 4.5 nM as determined by Scatchard analysis.

4-[(4-(1-Piperizinyl)phenyl)-aminocarbonyl]-1-piperidine-1-phenylacetic acid, which is the compound of this invention, inhibits [3H]-SK&F 107260 binding with a Ki of about 3.5 nM.

### Inhibition of Platelet Aggregation

Inhibition of platelet aggregation was determined following the procedure described in Nichols, et al., Thrombosis Research, 75, 143 (1994). Blood was drawn from the antecubital vein of normal human volunteers who had not taken a cyclooxygenase inhibitor within the previous 14 days into a plastic syringe containing one part 3.8% trisodium citrate to nine parts blood. Platelet rich plasma was prepared by centrifuging the blood at 200 g for 10 min at RT. The platelet rich plasma was drawn off and the remaining blood was centrifuged at 2400 g for 5 min at RT to make platelet poor plasma. Platelet count was measured with a model ZB1 Coulter Counter (Coulter Electronics Inc., Hialeah, Fla.) and was adjusted to 300,000/μl using platelet poor plasma. Platelet aggregation was studied in a Chrono-Log model 400VS Lumi Aggregometer (Chrono-Log, Havertown, Pa.) using platelet rich plasma stirred at 1200 r.p.m. and maintained at 37° C., with platelet poor plasma as the 100% transmission standard. Concentration-response curves for the ability of compounds to inhibit platelet aggregation, measured as the maximum change in light transmission, induced by a maximal concentration of adenosine diphosphate (10 μM) were constructed and the IC₅₀ was determined as the concentration of antagonist required to produce 50% inhibition of the response to the agonist.

### In Vivo Inhibition of Platelet Aggregation

In vivo inhibition of thrombus formation is demonstrated by recording the systemic and hemodynamic effects of infusion of the peptides into anesthetized dogs according to the methods described in Aiken et al., Prostaglandins, 19, 629 (1980).

The examples which follow are intended to in no way limit the scope of this invention, but are provided to illustrate how to make and use the compound of this invention. Many other embodiments will be readily apparent and available to those skilled in the art.

### General

Nuclear magnetic resonance spectra were obtained using either a Bruker AM 250 or Bruker AC 400 spectrometer. Chemical shifts are reported in parts per milliom (δ) downfield from the internal standard tetramethylsilane. Mass spectra were taken on either VG 70 FE or VG ZAB HF instruments using fast atom bombardment (FAB) or electrospray (ES) ionization techniques. Elemental analyses were performed by Quantitative Technologies Inc., Whitehouse, N.J.

E. Merck Silica Gel 60 F-254 thin layer plates were used for thin layer chromatography. Flash chromatography was carried out on E. Merck Kieselgel 60 (230-400 mesh) silica gel. Reverse phase flash chromatography was carried out on YMC-Gel (S20-120A) reverse phase silica gel. Radial chromatography was caried out on a Chrmoatotron (Model 8924; Harrison Research Company, Palo Alto, Calif.).

All other materials and solvents were obtained from commercial sources and were used without further purification.

### EXAMPLE 1

Preparation of 4-[(4-(1-piperizinyl)phenyl)aminocarbonyl]-1-piperidine-1-phenylacetic acid

a) 1-t-Butoxycarbonyl-4-(4-nitrophenyl)piperazine

To a solution of 1-(4-nitrophenyl)piperazine (2.50 g, 12.1 mmol) in THF (35 mL) at 0° C. was added portionwise di-t-butyldicarbonate (3.16 g, 14.5 mmol). The ice bath was removed and the reaction was allowed to warm to RT. After 1.5 h, the solvent was removed under reduced pressure and the residue was azeotroped with hexanes (3 times, to remove the t-BuOH) to give the 3.71 g of the desired product as a yellow powder. This was used in the next step without further purification. MS (ES+) m/z 308.2 (M+H⁺).

b) N-t-Butoxycarbonyl-isonipecotic acid, benzyl ester

To isonipecotic acid (1.00 g, 7.74 mmol) in THF (20 mL) and H₂O (10 mL) was added NaOH (0.93 g, 23.3 mmol). After the NaOH dissolved, the reaction was cooled in an ice bath and di-tbutyldicarbonate (2.53 g, 11.6 mmol) was added portionwise. The ice bath was removed and the reaction was allowed to stir at RT. After 18 h, the bulk of the solvent was removed under pressure and the residue was partitioned between H₂O and EtOAc. The aqueous layer was made acidic (pH=3) with 1N HCl and then extracted with EtOAc. The solvent was removed under reduced pressure and the residue was azeotroped from hexanes (3 times to remove the t-BuOH) to give 1.66 g of a white solid. Benzyl alcohol (2.40 mL, 23.2 mmol), EDC (2.23 g, 11.6 mmol) and DMAP (0.09 g, 0.74 mmol) were added to a solution of this material in CH₂Cl₂ (30 mL) at RT. After 5 days, the reaction was diluted with CH₂Cl₂ and washed with 1N HCl, sat. NaHCO₃ and brine and then dried over Na₂SO₄. Removal of solvent gave 3.35 g of the crude material which was purified by radial chromatography (20% EtOAc/hexanes, silica gel, 6 mm plate) to give 1.95 g of the desired product as a clear oil. ¹H NMR (400 MHz, CDCl₃) δ7.37−7.31 (m, 5H), 5.12 (s, 2H), 4.00 (m, 2H), 2.82 (m, 2H), 1.90 (m, 4H), 1.60 (m, 2H), 1.45 (s,

c) Ethyl 2-[4-(benzyloxycarbonyl)-1-piperidine]-phenylacetate

To the material obtained from Example 1(b) (0.80 g, 2.50 mmol) in CH₂Cl₂ (10 mL) was added trifluoroacetic acid (10 mL) at RT. When the gas evolution ceased (approx. 45 min.) the solvent was removed under vacuum. The residue was dissolved in THF (20 mL) and Et₃N (1.05 mL, 7.53 mmol) and ethyl alpha-bromophenylacetic acid (0.91 g, 3.74 mmol) was added. After heating the reaction at reflux for 18 h, the reaction was cooled to RT and the solvent was removed under reduced pressure. The product was isolated by radial chromatography (5% EtOAc/hexanes, silica gel, 6 mm plate) to give 0.84 g of the desired material as a pale yellow oil. ¹H NMR (400 MHz, CDCl₃) δ7.40 (m, 10H), 5.10 (s, 2H), 4.17 (m, 2H), 4.00 (s, 1H), 2.95 (m, 1H), 2.80 (m, 1H), 2.38 (m, 1H), 2.22 (m, 1H), 2.05−1.80 (m, 5H), 1.15 (m, 3H).

d) 4-[(4-(4-t-Butoxycarbonyl-1-piperizinyl)phenyl)aminocarbonyl]-1-piperidine-phenylacetic acid, ethyl ester

To the compound of Example 1(c) (0.84 g, 2.20 mmol) in EtOH (10 mL) was added 10% Pd/C (0.20 g). The reaction vessel was flushed with hydrogen and then fitted with a hydrogen filled balloon. After 1 h, the hydrogen was vented and the catalyst was removed by filtration through celite. The filter cake was rinsed with EtOH and the combined organic filtrates were concentrated to give 0.62 g of the desired product as a clear oil.

To the compound of Example 1(a) (0.72 g, 2 34 mmol) in EtOH (10 mL) was added 10% Pd/C (0.21 g). The reaction vessel was flushed with hydrogen and then fitted with a hydrogen filled balloon. After 2 h, the hydrogen was vented and the catalyst was removed by filtration through celite. The filter cake was rinsed with EtOH and the combined organic filtrates were concentrated to give 0.76 g of the desired product as a clear oil. This material was dissolved in pyridine (10 mL) and added to the material obtained above followed by EDC (0.49 g, 2.56 mmol). After 18 h at RT, the solvent was removed under reduced pressure and the product was isolated by flash chromatograpy (5% MeOH/CHCl₃, silica gel) to give 1.06 g of the desired product as a white foam. MS(ES+) m/z (551.4 (M+H⁺).

e) 4-[(4-(1-Piperizinyl)phenyl)aminocarbonyl]-1-piperidine-phenylacetic acid

To the compound of Example 1(d) (1.06 g, 1.93 mmol) in EtOH (6 mL) was added 1N NaOH (6 mL). After stirring at RT for 30 h, the reaction was quenched by acidifying to pH=5 with 1N HCl. The solvent was removed under reduced pressure and the residue was azeotroped from toluene 2 times. The residue was dissolved in CH₂Cl₂ (10 mL) and trifluoroacetic acid (10 mL). After 3 h at RT, the solvent was removed under reduced pressure to give a dark residue. Reverse phase flash chromatography (step gradient: H₂O; 10% CH₃CN/H₂O; 20% CH₃CN/H₂O) gave 0.23 g of the desired material as a white powder. MS (ES+) m/z 423.4 (M+H⁺). Anal. (C₂₄H₃₀N₄O₃.2CF₃CO₂H.2H₂O) calcd: C, 48.98; H, 5.28; N, 8.16. Found: C, 48.80; H, 4.80; N, 7.85.

### EXAMPLE 2

Preparation of (−)-4-[(4-(1-piperizinyl)phenyl)aminocarbonyl]-1-piperidine-1-phenylacetic acid

a) (−)-4-[(4-(1-Piperizinyl)phenyl)aminocarbonyl]-1-piperidine-1-phenylacetic acid

The compound of Example 1(d) (0.35 g) was resolved by preparative HPLC (Chiralpak AD, 90:10:0.1:0.1 hexane/ethanol/trifluoroacetic acid/diethylamine) to give 0.12 g one enantiomer of the compound of Example 1(d). HPLC t_{R}8.8 min (Chiralpak AD, 4.6×250 mm, 1.0 mL/min, 90:10:0.1:0.1 hexane/ethanol/trifluoroacetic acid/diethylamine, UV detection at 220 nm).

In a manner analogous to Example 1(e), the material prepared above gave 40 mg of the desired material as an off-white powder.

Anal. (C₂₄H₃₀N₄O₃.3CF₃CO₂H.2.5H₂O) calcd: C, 44.51; H, 4.73; N, 6.92.

Found: C, 44.60; H, 4.48; N, 6.89.

### EXAMPLE 3

Preparation of (+)-4-[(4-(1-piperizinyl)phenyl)aminocarbonyl]-1-piperidine-1-phenylacetic acid

a) (+)-4-[(4-(1-Piperizinyl)phenyl)aminocarbonyl]-1-piperidine-1-phenylacetic acid

The compound of Example 1(d) (0.35 g) was resolved by preparative HPLC (Chiralpak AD, 90:10:0.1:0.1 hexane/ethanol/trifluoroacetic acid/diethylamine) to give 0.18 g one enantiomer of the compound of Example 1(d). HPLC t_{R}8.8 min (Chiralpak A D, 4.6×250 mm, 1.0 mL/min, 90:10:0.1:0.1 hexane/ethanol/trifluoroacetic acid/diethylamine, UV detection at 220 nm).

In a manner analogous to Example 1(e), the material prepared above gave 60 mg of the desired material as an off-white powder.

Anal. (C₂₄H₃₀N₄O₃.4CF₃CO₂H.1H₂O) calcd: C, 42.78; H, 3.99; N, 6.23

Found: C, 42.87; H, 4.05; N, 6.25.

### EXAMPLE 4

Preparation of 2-(4-[4-(1-piperazinyl)phenylamino]carbonylpiperadino)-2-(4-methoxyphenyl)acetic acid

a) Methyl 2-[4-(benzyloxycarbonyl)piperadino]-2-(4-methoxyphenyl)acetate

In a manner analogous to Example 1(c), the material from Example 1(b) and methyl 2-bromo-2-(4-methoxyphenyl)acetate gives methyl 2-[4-(benzyloxycarbonyl)piperadino]-2-(4-methoxyphenyl)acetate

b) 2-(4-[4-(4-tert-Butoxycarbonyl-1-piperazinyl)phenylamino]carbonylpiperadino)-2-(4-methoxyphenyl)acetic acid, methyl ester

In a manner analogous to Example 1(d), the compound of Example 4(a) and the compound of Example 1(a) gives 4-[(4-(4-t-butoxycarbonyl-1-piperizinyl)phenyl)aminocarbonyl]-1-piperidine-1-(4-methoxyphenyl)acetic acid, methyl ester.

c) 2-(4-[4-(1-piperazinyl)phenylamino]carbonylpiperadino)-2-(4-methoxyphenyl)acetic acid

In a manner analogous to the preparation of Example 1(e), the compound of Example 4(b) gives the title compound.

### EXAMPLE 5

Preparation of 2-(4-[4-(1-piperazinyl)phenylamino]carbonylpiperadino)-2-(4-tert-butylphenyl)acetic acid

In a manner analogous to Example 4, methyl 1-bromo-1-(4-tert-butylphenyl)acetate gives the title compound.

### EXAMPLE 6

Preparation of 2-(4-[4-(1-piperazinyl)phenylamino]carbonylpiperadino)butanoic acid

In a manner analogous to Example 4, methyl 2-bromobutanoate gives the title compound.

### EXAMPLE 7

Preparation of 3-methyl-2-(4-[4-(1-piperazinyl)phenylamino]-carbonylpiperadino)butanoic acid

In a manner analogous to Example 4, methyl 2-bromo-3-methylbutanoate gives the title compound.

### EXAMPLE 8

Preparation of 2-(4-[4-(1-piperazinyl)phenylamino]carbonylpiperadino)-2-(4-nitrophenyl)acetic acid

a) N-t-Butoxycarbonylisonipecotic Acid, Allyl Ester

Sodium hydroxide is added to isonipecotic acid in THF and water. After the NaOH dissolves, the reaction is cooled in an ice bath and di-t-butyldicarbonate is added portionwise. After stirring at RT for 18 h, the bulk of the solvent is removed under reduced pressure and the residue is partitioned between H₂O and EtOAc. The aqueous layer is made acidic (pH=3) with 1N HCl and is extracted with EtOAc. The solvent is removed under reduced pressure and the residue is azeotroped from hexanes (3 times). This material is dissolved in CH₂Cl₂ and allyl alcohol, EDC and DMAP are added. After 18 h, the reaction is diluted with CH₂Cl₂ and is washed with 1N HCl, sat. NaHCO₃ and dried over Na₂SO₄. The solvent is removed under reduced pressure and the material is purified by flash chromatography.

b) Methyl 2-[4-(allyloxycarbonyl)-1-piperadinyl]-2-(4-nitrophenyl)acetate

The material from Example 8(a) is dissolved in CH₂Cl₂ and trifluoroacetic acid is added. The reaction is stirred at RT until the gas evolution ceases. The solvent is removed under reduced pressure and the residue is dissolved in THF and Et₃N. Methyl 2-bromo-2-(4-nitrophenyl)acetate is added and the reaction is heated at reflux for 18 h. The solvent is removed under reduced pressure after allowing the reaction to cool to RT. The desired material is isolated by flash chromatography.

c) 2-(4-[4-(4-t-Butoxycarbonyl-1-piperazinyl)phenylamino]carbonylpiperadino)-2-(4-nitrophenyl)acetic acid, methyl ester

The compound of Example 8(b) is dissolved in 10% N-methylaniline in DMF. Tetrakistriphenylphosphine palladium is added and the reaction is heated to 60° C. When the reaction is complete, it is allowed to cool to RT and filtered through a pad of celite. The solvent is removed under vacuum.

A hydrogenation vessel is charged with EtOH, 10% Pd/C and the compound from Example 1(a). The reaction vessel is fitted with a hydrogen-filled balloon. After 2h, the hydrogen is vented and the catalyst is removed by filtration through a bed of celite. The solvent is removed by filtration and the residue is dissolved in pyridine. The material obtained above and EDC are added to the pyridine solution. After 1 8h, the solvent is removed under reduced pressure and the product is isolated by flash chromatography.

d) 2-(4-[4-(1-Piperazinyl)phenylamino]carbonylpiperadino)-2-(4-nitrophenyl)acetic acid

In a manner analogous to the preparation of Example 1(e), the compound of Example 8(c) gives the title compound.

### EXAMPLE 9

Oral Dosage Unit Composition

A capsule for oral administration is prepared by mixing and milling 50 mg of the compound of Example 1 with 75 mg of lactose and 5 mg of magnesium stearate. The resulting powder is screened and filled into a hard gelatin capsule.

### EXAMPLE 10

Oral Dosage Unit Composition

A tablet for oral administration is prepared by mixing and granulating 20 mg of sucrose, 150 mg of calcium sulfate dihydrate and 50 mg of the compound of Example 1 with a 10% gelatin solution. The wet granules are screened, dried, mixed with 10 mg starch, 5 mg talc and 3 mg stearic acid; and compressed into a tablet.

The foregoing is illustrative of the making and using of this invention. This invention, however, is not limited to the precise embodiments described herein, but encompasses all modifications within the scope of the claims which follow. The various references to journals, patents and other publications which are cited herein comprise the state of the art and are incorporated herein by reference as though fully set forth.

What is claimed is:

1. A compound which is 4-[(4-(1-piperizinyl)phenyl)aminocarbonyl]-1-piperidine-1-phenylacetic acid or a pharmaceutically acceptable salt thereof.

2. A compound which is (−)-4-[(4-(1-piperizinyl)phenyl)-aminocarbonyl]-1-piperidine-1-phenylacetic acid or (+)-4-[(4-(1-piperizinyl)phenyl)aminocarbonyl]-1-piperidine-1-phenylacetic acid;
or a pharmaceutically acceptable salt thereof.

3. A compound which is:
2-(4-[4-(1-piperazinyl)phenylamino]carbonylpiperadino)-2-(4-methoxyphenyl)acetic acid;
2-(4-[4-(1-piperazinyl)phenylamino]carbonylpiperadino)-2-(4-tert-butylphenyl)acetic acid;
2-(4-[4-(1-piperazinyl)phenylamino]carbonylpiperadino)butanoic acid;
3-methyl-2-(4-[4-(1-piperazinyl)phenylamino]carbonylpiperadino)butanoic acid; or
2-(4-[4-(1-piperazinyl)phenylamino]carbonylpiperadino)-2-(4-nitrophenyl)acetic acid;
or a pharmaceutically acceptable salt thereof.

4. A pharmaceutical composition comprising a compound according to any one of claims 1-3 and a pharmaceutically acceptable carrier.

5. A method for effecting inhibition of platelet aggregation which comprises administering a compound according to any one of claims 1-3.

6. A method for treating stroke or a transient ischemia attack or myocardial infarction which comprises administering a compound according to any one of claims 1-3.

7. A method for promoting reperfusion of an artery or vein and inhibiting reocclusion which comprises administering a fibrinolytic agent and a compound according to any one of claims 1-3.

8. A compound according to any one of claims 1 to 3 for use as a medicament.

9. The use of a compound as defined in any one of claims 1 to 3 in the manufacture of a medicament for the inhibition of platelet aggregation.

10. The use of a compound as defined in any one of claims 1 to 3 in the manufacture of a medicament for the treatment of stroke, a transient ischemia attack or myocardial infarction.

11. The use of a compound as defined in any one of claims 1 to 3 and a fibrinolytic agent in the manufacture of a medicament for promoting reperfusion of an artery or vein and inhibiting reocclusion.

12. A compound of the formula (II):

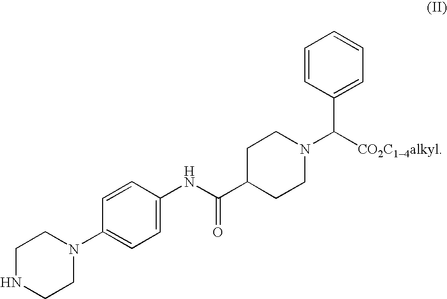

13. A process for preparing a compound of claim 1, which process comprises reacting a compound of formula (III) with a compound of formula (IV): 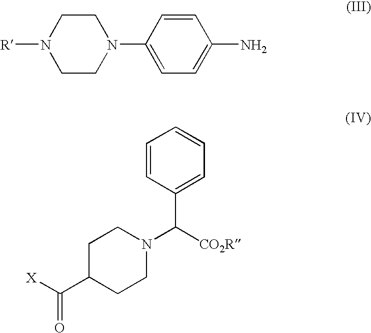
wherein R′ is an amine protecting group, R″ is a C₁₋₄alkyl group and X is OH or chloro;
and thereafter removing any protecting groups, and optionally forming a pharmaceutically acceptable salt.
